(19) United States
(12) Patent Application Publication
(10) Pub. No.: US 2024/0110128 A1
(43) Pub. Date: Apr. 4, 2024

(54) ALKOXYLATES HAVING IMPROVED HYDROTROPIC POWER

(71) Applicant: Arkema France, Colombes (FR)

(72) Inventors: Carl Bouret, Chateauroux (FR); Jean-Philippe Gillet, Pierre-Benite Cedex (FR); Tony Bartolini, Colombes Cedex (FR)

(73) Assignee: Arkema France, Colombes (FR)

(21) Appl. No.: 17/769,135

(22) PCT Filed: Oct. 16, 2020
(86) PCT No.: PCT/FR2020/051859

§ 371 Date: Apr. 14, 2022

(30) Foreign Application Priority Data
Oct. 18, 2019 (FR) FR1911678

(51) Int. Cl. C11D 1/722

(52) U.S. Cl. CPC C11D 1/722; C11D 2111/10

(57) ABSTRACT

The invention relates to detergent formulations comprising at least one secondary alcohol alkoxylate, and to the use of said at least one secondary alcohol alkoxylate as a surfactant having an improved hydrotropic power.

### CROSS-REFERENCE TO RELATED APPLICATIONS

This application is the U.S. national phase of International Application No. PCT/FR2020/051859, filed 16 Oct. 2020, which claims priority to French Application No. FR 1911678, filed 18 Oct. 2019, the disclosure of each of these applications being incorporated herein by reference in its entirety for all purposes.

### FIELD OF THE INVENTION

The present invention relates to the general field of formulations containing surfactants, more particularly to that of formulations containing nonionic surfactants, and especially to that of formulations containing alkoxylated nonionic surfactants.

### BACKGROUND OF THE INVENTION

Alkoxylated compounds are nowadays known to represent a class of compounds that offers a broad range of properties, with multiple applications, such as solvents, hydrotropic agents or else surface-active agents, to name only a few of these applications. Alkoxylated compounds therefore constitute a class of compounds having surfactant properties that display a true industrial interest in a great number of fields of application.

The uses of alkoxylated compounds as surfactants constitute one very much favored application with the volumes of surfactants being nowadays substantial for very numerous and varied uses, and, for example, in the form of formulations for detergency or cleaning or washing, to give but a few examples.

Within detergent formulations, the intrinsic properties of the surfactants allow them to bring about compatibility between the soiling, which is usually organic and lipophilic, and a hydrophilic medium, generally an aqueous medium, and so to enable the removal of said lipophilic soiling with the hydrophilic medium.

Such surfactants may, however, exhibit problems of solubility with the media in which they are used, such that it is often necessary to add one or more solubilizers for the surfactants. Among particularly suitable solubilizers, use is made generally of hydrotropic agents.

A hydrotropic agent, indeed, is a compound which solubilizes the hydrophobic compounds in aqueous solutions. Hydrotropic agents are now commonly used industrially in the formulation of detergents particularly to make it possible to have a greater concentration of surfactants.

It is known, moreover, that certain surfactants themselves possess a hydrotropic power, so making the addition of another hydrotropic agent optional or even superfluous. It may thus prove advantageous to have surfactants with enhanced hydrotropic power, or even high hydrotropic power, so as to avoid the addition of other hydrotropic agents, so as not to complicate the detergent formulation formulas, to be able to lower the cost of producing them, but also and especially, to avoid further loading the formulations, for obvious reasons of environmental protection.

This need is particularly acute for those detergent formulations which comprise alkoxylated nonionic surfactants, in other words those having at least one alkoxy unit and preferably at least two. These formulations include, by way of illustration but not of limitation, multi-purpose cleaners, cosmetic products, formulations for hard surface cleaning, the cleaning of garments, the cleaning of sanitaryware, the washing of automobiles in general, cleaning in place (CIP), and others.

Alkoxylated nonionic surfactants of these kinds are described for example in international patent application WO2009000852, said surfactants being alkoxylates of primary Neodol alcohols (branched polyalcohols obtained by Fischer-Tropsch process).

Further polyalkoxylates are described in international patent application WO2012005897, which discloses the alkoxylation of alcohols for use as surfactants in diverse applications.

Alkoxylated nonionic surfactants, such as for example those described in the documents cited above, nevertheless exhibit hydrotropic powers which are often insufficient, and consequently today there remains a need for detergent formulations based on nonionic surfactants with further enhanced or even substantial hydrotropic powers.

Another objective of the present invention is that of providing detergent formulations based on surfactants with enhanced hydrotropic power which are obtained from biobased products, and more particularly from biobased products which are not in competition with the human or animal food chain.

It has now been found that these objectives may be achieved, in their entirety or at least in part, by virtue of the invention which is set out in the description hereinafter. Still further advantages will become apparent in said description of this invention.

The Applicant has now found, surprisingly, that some alkoxylates exhibit enhanced hydrotropic power relative to that observed with alkoxylates known in the prior art, and that these alkoxylates may advantageously form part of the composition of detergent formulations, as a surfactant.

### SUMMARY OF THE INVENTION

Therefore, and according to a first aspect, the present invention relates to a detergent formulation comprising at least one secondary alcohol alkoxylate, wherein said secondary alcohol comprises from 3 to 22 carbon atoms, preferably from 5 to 22 carbon atoms, more preferably from 5 to 20 carbon atoms, very preferably from 5 to 18 carbon atoms, endpoints included, and said secondary alcohol is alkoxylated with oxyalkylene units, chosen from oxyethylene (OE), oxypropylene (OP), and oxybutylene (OB), the total number of oxyalkylene units being between 2 and 100, preferably between 3 and 100, more preferably between 3 and 50, with further preference between 3 and 40, and very particularly between 3 and 30, endpoints included.

The oxyalkylene units of the secondary alcohol alkoxylate may be identical or different and, if they are different, they may be arranged in any way whatsoever: for example, randomly, in blocks, in alternation or sequenced, or other. For the purposes of the present invention, particular preference is given to using a secondary alcohol alkoxylate as defined above which possesses oxyalkylene units arranged in blocks.

According to another preferred aspect of the invention, the alcohol used as starting substrate for the alkoxylation reaction or reactions has a degree of branching of 0, 1 or 2, more preferably 1 or 2.

### DETAILED DESCRIPTION OF THE INVENTION

In the sense of the present invention, the expression “degree of branching” denotes the total number of terminal methyl (—CH₃), methylene (═CH₂) and methyne (═CH) groups (referred to as “end groups”) present on the alcohol used as starting substrate for the alkoxylation reaction or reactions, with the value 1 being subtracted from this total number. In other words, the degree of branching, D, is an integer which is equal to the difference between the sum total of the end groups present on the alcohol used as starting substrate for the alkoxylation reaction or reactions, and 1. This equation may be expressed as follows:

D=Σ(end groups)−1

Accordingly, if the starting alcohol comprises 2 terminal methyl groups, the degree of branching is then 1 (D=Σ(end groups)−1=2−1=1). For example, the degree of branching of cardanol (1 terminal methylene group) is 0, that of 2-octanol is 1, and that of 4-methyl-2-pentanol is 2.

As indicated above, the alcohol used as a starting substrate for the alkoxylation reaction or reactions comprises from 3 to 22, preferably from 5 to 22, carbon atoms, more preferably from 5 to 20 and very preferably from 5 to 18 carbon atoms. The carbon atoms may be in a linear, branched or wholly or partly cyclic chain. According to one preferred embodiment, in the formulation according to the present invention, the secondary alcohol has a weight-average molar mass of from 45 g mol⁻¹ to 300 g mol⁻¹, preferably from 70 g mol⁻¹ to 250 g mol⁻¹, more preferably from 80 g mol⁻¹ to 200 g mol⁻¹, endpoints included.

The secondary alcohol used as starting substrate and intended for alkoxylation may be of any type and any origin, and more particularly of petroleum origin, or of biobased origin, such as of plant or animal origin, for example. Preference, however, is given to a secondary alcohol of biobased origin, for obvious environmental protection reasons.

Preference is given, moreover, to a secondary alcohol containing from 3 to 14 carbon atoms, more preferably from 6 to 12 carbon atoms, and, according to one particularly preferred embodiment, the secondary alcohol is chosen from 2-octanol and 4-methyl-2-pentanol, and very advantageously the secondary alcohol is 2-octanol.

The reason is that 2-octanol is of particular interest in a number of respects, in particular because it comes from a biobased product which is not in competition with human or animal food. Moreover, 2-octanol which has a high boiling point, is biodegradable and exhibits a good ecotoxicological profile.

As indicated above, the repeating alkoxy units are chosen from ethylene oxide, propylene oxide and butylene oxide units and mixtures thereof. The term “ethylene oxide unit” refers to a unit obtained from ethylene oxide after opening of the oxirane ring, “propylene oxide unit” refers to a unit obtained from propylene oxide after opening of the oxirane ring, and “butylene oxide unit” refers to a unit obtained from butylene oxide after opening of the oxirane ring.

The abovementioned alkylene oxides may be of diverse origins, and more particularly “mass balance” alkylene oxides, more particularly “mass balance ethylene oxide” and alkylene oxides of biobased origin. The ethylene oxide is advantageously biobased in origin; for example, the ethylene oxide may be obtained by oxidation of biobased ethylene from the dehydration of bioethanol, itself originating from corn starch, from lignocellulosic materials, from agricultural residues such as sugar cane bagasse, for example, and others.

The secondary alcohol alkoxylate which has now been defined and is useful in the preparation of detergent formulations with enhanced hydrotropic power in accordance with the present invention may be prepared by any means known to those skilled in the art, and more particularly by any known method of alcohol alkoxylation.

Alcohol alkoxylation is conventionally and advantageously performed in the presence of a catalyst, the alkoxylation being advantageously performed by basic or alkaline catalysis, using, for example, sodium hydroxide (NaOH) or potassium hydroxide (KOH), referred to as soda or potash catalysis respectively.

Other types of catalysts may be used, and particularly those now known by the person skilled in the art who is an alkoxylation specialist to result in alkoxylates having a narrow or even very narrow numerical distribution of alkoxylate units. Catalysts of this kind are known as “narrow range” (narrow distribution) catalysts, and are chosen, for example, from catalysts based on calcium or based on boron derivatives (such as the acidic BF₃ derivative catalysts), hydrotalcite catalysts, and dimetallic cyanide (DiMetallic Cyanide or DMC) catalysts.

For the purposes of the present invention, preference is given to those formulations in which said at least one secondary alcohol alkoxylate is chosen from narrow range secondary alcohol alkoxylates, and especially those obtained by alkoxylation of a secondary alcohol via narrow range catalysis, and more preferably via DMC catalysis.

According to one embodiment, the secondary alcohol alkoxylate used as a surfactant with enhanced hydrotropic power in the detergent formulation according to the present invention is a capped secondary alcohol alkoxylate, this being an alkoxylate in which the terminal —OH moiety is substituted, as described for example in document EP2205711 or else in international patent application WO2004037960.

According to one preferred aspect, the substituent of the terminal moiety, also called the cap of the terminal moiety, or more simply endcap, is a group chosen from linear or branched alkyls comprising from 1 to 6 carbon atoms, the phenyl group, the benzyl group, hydrocarbon groups bearing a carboxyl function —COO—, and groups bearing a sugar unit.

The endcap of the secondary alcohol alkoxylate is preferably chosen from methyl, ethyl, propyl, butyl, benzyl and alkylcarboxyl groups and salts of the latter. The possible salts of the carboxyl function include the salts well known to those skilled in the art and more particularly metal salts, alkaline metal salts, alkaline earth metal salts and ammonium salts, to mention only the main representatives thereof. Particularly preferred salts are the sodium, potassium, calcium and ammonium salts.

According to another embodiment, the endcap of the secondary alcohol alkoxylate is chosen from alkylenecarboxyls and salts thereof, optionally functionalized. One typical, nonlimiting example is represented by the sulfosuccinate group, and more particularly sodium sulfosuccinate, potassium sulfosuccinate, calcium sulfosuccinate and ammonium sulfosuccinate.

According to yet another embodiment, the endcap of the secondary alcohol alkoxylate is chosen from groups bearing a sugar unit, for instance glucose (in the case of monoglucosides) or bearing two or more sugar units (in the case of alkylpolyglucosides, also called APGs).

According to yet another embodiment of the present invention, preference is given to detergent formulations in which said at least one secondary alcohol alkoxylated comprises at least ethylene oxide (EO) units. According to another preferred embodiment of the present invention, the detergent formulation comprises at least one secondary alcohol alkoxylate having at least ethylene oxide (EO) units and at least propylene oxide (PO) units, where said units may be distributed randomly, in alternation or in blocks, and preferably in blocks.

According to yet another preferred embodiment, the total number of repeating units borne by the secondary alcohol alkoxylate in the detergent formulation according to the present invention is, with endpoints included, between 1 and 30, preferably between 2 and 20, more preferably between 3 and 20, advantageously between 3 and 15.

In one especially preferred embodiment, the detergent formulation according to the present invention comprises at least one 2-octanol alkoxylate. With particular advantage, said alkoxylate is chosen from 2-octanol having ethoxy units, 2 octanol having ethoxy and propoxy units, 2-octanol having ethoxy and butoxy units, 2-octanol having propoxy units, 2-octanol having propoxy and butoxy units, and 2-octanol having butoxy units. Preference, however, is given to detergent formulations comprising at least one 2-octanol having ethoxy units or at least one 2-octanol having ethoxy and propoxy units.

Examples of secondary alcohol alkoxylates especially suitable for the formulation according to the present invention are those chosen from 2-octanol 2-15 EO, 2-octanol 2-15 EO 1 PO, 2-octanol 2-15 EO 1-15 BO, 2-octanol 2-15 EO 1-15 PO, and 2 octanol 1-6 EO 1-15 PO.

The amount of secondary alcohol alkoxylate(s) in the detergent formulation according to the present invention may vary within wide proportions according to the nature of the one or more alkoxylates and the nature and intended use of the formulation. As a general rule, the amount of secondary alcohol alkoxylate(s) is between 1% and 99%, more generally between 1% and 50%, advantageously between 1% and 25%, by weight of alkoxylate(s) relative to the total weight of the formulation.

The formulation according to the present invention may comprise any type of additives or fillers known to the person skilled in the art who is a formulations specialist and particularly a detergent formulations specialist.

Accordingly, and by way of nonlimiting examples, the detergent formulation according to the present invention may comprise one or more of the additives and fillers chosen from detergency agents, especially basic detergency agents, such as sodium hydroxide, for example, surfactants, fragrances, dyes, inert fillers, impregnating agents, aqueous, organic and aqueous-organic solvents chosen from water, alcohols, glycols and polyols, mineral oils, vegetable oils, waxes, and others, alone or in mixtures of two or more of them, in any proportions.

The formulation according to the invention may more particularly comprise one or more additives and fillers that are well known to those skilled in the art, such as, for example, and without limitation, anionic, cationic, amphoteric and nonionic surfactants, rheology modifiers, demulcents, foaming agents, antifoam agents, hydrotropic agents, antideposition agents, dispersants, pH control agents, dyes, antioxidants, preservatives, corrosion inhibitors, biocides, and other additives, such as, for example, products containing sulfur, boron, nitrogen, phosphorus, and others. The nature and amount of the additives and fillers may vary within wide proportions depending on the nature of the intended application and may readily be adapted by a person skilled in the art.

As indicated above, the detergent formulation according to the present invention comprises a secondary alcohol alkoxylate as has now been defined, with enhanced hydrotropic power, thereby endowing the formulation comprising it with entirely advantageous properties, especially in terms of solubilization but also in terms of detergent power. It has been observed, indeed, that the formulations of the present invention, and more particularly those comprising a 2-octanol alkoxylate, exhibit an enhanced detergent power, owing in particular to the high hydrotropic power of the secondary alcohol alkoxylate.

The present invention relates, lastly, to the use of at least one secondary alcohol alkoxylate as a surface-active agent with enhanced hydrotropic power in a detergent formulation. It has been observed, indeed, that the secondary alcohol alkoxylates of the kind which have now been defined, and more particularly the alkoxylates obtained by narrow range catalysis, exhibit enhanced hydrotropic power, their hydrotropic power in particular being enhanced relative to that of other alkoxylates having the same number of alkoxy units but a different substrate, such as a primary alcohol, for example.

The secondary alcohol alkoxylates as have now been defined, and especially those obtained by narrow range catalysis, have highly interesting application properties in terms of performance and have very advantageous biodegradability profiles, especially for low levels of alkoxylation, ≤8 units, preferably <8 units, more preferably ≤6 units, and more preferably still ≤4 units.

The secondary alcohol alkoxylates as have now been defined, and especially those obtained by narrow range catalysis, thus find entirely appropriate applications by virtue of their very good degreasing, solubilizing and emulsifying properties, and more particularly their enhanced hydrotropic power, so making them surfactants of choice when they are used in detergent formulations for dishwashing. The reason is that enhanced hydrotropic power leads generally to better results in terms of “spotting”, referring to marks left on the dishware and more particularly on glass.

Accordingly, the secondary alcohol alkoxylates as have now been defined find entirely advantageous applications in detergent formulations, and more particularly in detergent formulations for dishwashing, for multi-purpose cleaning (cleaners), for hard surface cleaning, for cleaning garments (laundry), for cosmetic products, for cleaning sanitaryware, for washing automobiles in general, for cleaning in place (CIP), and others.

The secondary alcohol alkoxylates of the kind defined above may further be used in diverse formulations in which such surfactants are needed or desired as an emulsifier, wetting agents, solvents, or adjuvants, and more particularly in formulations for ore flotation, for metalworking fluids, for bituminous applications, for de-inking, for enhanced oil and gas recovery applications, for protection from corrosion, for hydraulic fracturing, for soil remediation, in agrochemistry (for example, coatings on granular products, especially fertilizers and plant health products), but also as an antifoam agent, antistatic agent, paint adjuvant, textiles adjuvant, for polyols, and for production of electrodes and electrolytes for batteries, to state only the main fields of application.

The invention is now illustrated by the examples that follow, which are not in any way limiting.

### EXAMPLES

Secondary alcohol alkoxylates having variable numbers of alkoxy units are tested for their hydrotropic power and compared with primary alcohol alkoxylates having variable numbers of alkoxy units. The alkoxylates are prepared by techniques known to those skilled in the art. Unless otherwise indicated, the alkoxylates are prepared by narrow range catalysis, according to the procedure described in WO2019092366 A1, for example.

The hydrotropic power of a test product is characterized by heating a solution containing a reference product and noting the temperature at which the haze formed owing to desolubilization of the reference product in the solution disappears. The hydrotropic power corresponds to this temperature (expressed in ° C.). The higher this temperature, the more hydrotropic the product.

A solution is heated in a beaker until a persistent haze appears, said solution containing (percentages by weight unless otherwise indicated):
5% of Surfaline® OX1308L (ethoxylated isotridecanol containing 8 EO) diluted with 15% (by weight) of water, sold by Arkema,
2% of sodium hydroxide,
1% of the test product,
q.s. 100% demineralized water.

This is left to cool naturally and the temperature at which the haze disappears is noted. The hydrotropic power corresponds to this temperature (expressed in ° C.). The results are presented in table 1 below:

TABLE 1

| | No. | | No. | Hydrotropic |
|---|---|---|---|---|
| | carbon | Type of | alcoxy | power |
| structure | atoms | alcohol | units | (° C.) |
| 2-octanol* + 6 EO + 4 PO | 8 | secondary | 6 + 4 | 33.9 |
| C₈ alcohol + 4 EO | 8 | primary | 4 | 28.4 |
| C₉C₁₁ alcohol + 4 EO | 9-11 | primary | 4 | 24.3 |
| C₉C₁₁ alcohol + 5.5 EO | 9-11 | primary | 5.5 | 29.9 |
| C₁₀ Guerbet alcohol + 3.3 EO | 10 | primary | 3.3 | 20 |
| C₁₀ Guerbet alcohol + 5 EO | 10 | primary | 5 | 28.3 |
| C₁₀ alcohol + 4 EO | 10 | primary | 4 | 24.8 |
| C₁₀ alcohol + 4 EO (catal. KOH) | 10 | primary | 4 | 27.4 |
| C₁₀ alcohol + 8 EO (catal. KOH) | 10 | primary | 8 | 37.7 |
| C₁₀ alcohol + 3 EO (catal. NaOH) | 10 | primary | 3 | 20 |
| C₁₀ alcohol + 4 EO (catal. NaOH) | 10 | primary | 4 | 22 |
| C₁₀ alcohol + 5 EO (catal. NaOH) | 10 | primary | 5 | 29.8 |
| C₁₀ alcohol + 8 EO (catal. NaOH) | 10 | primary | 8 | 38 |
| C₁₀ oxo alcohol + 3 EO (catal. NaOH) | 10 | primary | 3 | 23 |
| C₁₀ oxo alcohol + 4 EO (catal. NaOH) | 10 | primary | 4 | 29.5 |
| C₁₀ oxo alcohol + 5 EO (catal. NaOH) | 10 | primary | 5 | 29.5 |
| C₁₀ oxo alcohol + 6 EO (catal. NaOH) | 10 | primary | 6 | 35.5 |
| C₁₂C₁₄ alcohol + 5 EO + 4 PO | 12-14 | primary | 5 + 4 | 29 |
| (catal. KOH) | | | | |
| C₁₃ oxo alcohol + 8 EO (catal. NaOH) | 13 | primary | 8 | 34.3 |

*Oleris ® 2-octanol, of “refined” grade (purity >99%), sold by Arkema France.

These results show that for an identical number of alkoxy units, the secondary alcohol alkoxylates always have a higher hydrotropic power. This is particularly advantageous in the case of aqueous detergent formulations, which are usually basic aqueous formulations.

The present invention hence provides a simple and effective solution enabling in particular an increase in the concentration of surfactants in detergent formulations, while retaining a clear and perfectly homogeneous appearance, this solution being to add at least one secondary alcohol alkoxylate to said detergent formulation.

1. A detergent formulation comprising at least one secondary alcohol alkoxylate, wherein the secondary alcohol comprises from 3 to 22 carbon atoms, endpoints included, and the secondary alcohol is alkoxylated with oxyalkylene units, chosen from oxyethylene (OE), oxypropylene (OP), and oxybutylene (OB), the total number of oxyalkylene units being between 2 and 100, endpoints included.

2. The formulation as claimed in claim 1, wherein the secondary alcohol has a weight-average molar mass of from 45 g mol⁻¹ to 300 g mol⁻¹, endpoints included.

3. The formulation as claimed in claim 1, wherein the secondary alcohol comprises from 3 to 14 carbon atoms.

4. The formulation as claimed in claim 1, wherein the at least one secondary alcohol alkoxylate is chosen from narrow range secondary alcohol alkoxylates.

5. The formulation as claimed in claim 1, wherein the at least one secondary alcohol alkoxylate comprises at least ethylene oxide units (EO), where the units may be distributed randomly, in alternation or in blocks.

6. The formulation as claimed in claim 1, wherein the at least one secondary alcohol alkoxylate is chosen from 2-octanol having ethoxy units, 2 octanol having ethoxy and propoxy units, 2-octanol having ethoxy and butoxy units, 2-octanol having propoxy units, 2-octanol having propoxy and butoxy units, and 2-octanol having butoxy units.

7. The formulation as claimed in claim 1, wherein the at least one secondary alcohol alkoxylate is chosen from 2-octanol 2-15 EO, 2-octanol 2-15 EO 1 PO, 2-octanol 2-15 EO 1-15 BO, 2-octanol 2-15 EO 1-15 PO, and 2 octanol 1-6 EO 1-15 PO.

8. The formulation as claimed in claim 1, wherein the amount of secondary alcohol alkoxylate(s) is between 1% and 99% by weight of alkoxylate(s) relative to the total weight of the formulation.

9. The formulation as claimed in claim 1, further comprising one or more of the additives and fillers chosen from detergency agents, surfactants, fragrances, dyes, inert fillers, impregnating agents, aqueous, organic and aqueous-organic solvents chosen from water, alcohols, glycols and polyols, mineral oils, vegetable oils, waxes, and others, alone or in mixtures of two or more of them, in any proportions.

10. The use of at least one secondary alcohol alkoxylate as defined in claim 1 as a surface-active agent with hydrotropic power in a detergent formulation.

11. (canceled)

12. The use as claimed in claim 10, wherein the at least one secondary alcohol alkoxylate is obtained by narrow range catalysis.

13. The use as claimed in claim 10 in a detergent formulation for dishwashing, for multi-purpose cleaning, for hard surface cleaning, for garment cleaning, for cosmetic products, for cleaning sanitaryware, for washing automobiles in general, and for cleaning in place (CIP).
